(19) United States
(12) Patent Application Publication
(10) Pub. No.: US 2011/0301672 A1
(43) Pub. Date: Dec. 8, 2011

(54) UV-EMITTING DISCHARGE LAMP

(75) Inventors: Thomas Juestel, Witten (DE); Georg Greuel, Roetgen (DE)

(73) Assignee: KONINKLIJKE PHILIPS ELECTRONICS N.V., EINDHOVEN (NL)

(21) Appl. No.: 13/202,640

(22) PCT Filed: Feb. 17, 2010
(86) PCT No.: PCT/IB10/50701

§ 371 Date: Aug. 22, 2011

(30) Foreign Application Priority Data
Feb. 25, 2009 (EP) 09153675.5

(51) Int. Cl. A61N 5/06; G01J 3/10; A61L 2/10; H01J 1/63

(52) U.S. Cl. 607/90; 313/484; 250/504 R; 250/492.1

(57) ABSTRACT

The invention relates to a UV-B emitting discharge lamp comprising Pr(III) as an activator.

### FIELD OF THE INVENTION

The present invention is directed to novel materials for light emitting devices, especially to the field of novel materials for discharge lamps emitting UV radiation.

### BACKGROUND OF THE INVENTION

Fluorescent lamps which comprise an UV emitting phosphor are widely applied for cosmetic and medical purposes. These lamps usually generate UV light by e.g. utilizing an Hg low-pressure discharge and a luminescent screen comprising UV-B or UV-A phosphors or a blend of several UV-A/B phosphors. The most commonly applied phosphors are LaPO₄:Ce, SrAl₁₂O₁₉:Ce,Na, or LaB₃O₆:Bi,Gd as UV-B emitter and (Y,Gd)PO₄:Ce, BaSi₂O₅:Pb, or SrB₄O₇:Eu as UV-A emitter.

However, many of the presently applied low-pressure discharge lamps suffer the drawback of short-term degradation. This is caused by the interaction between the luminescent material and e.g. Hg ions from the discharge. The result of this chemical interaction is the formation of a black layer on top of the phosphor layer, resulting in phosphor greyishing and thus efficiency reduction. Degradation processes have also been observed in Xe excimer discharge lamps.

An excimer discharge lamp is a discharge lamp in which at least 1 component of the discharge-sustaining filling forms an excimer during the lamp operation. The excimer forming is essential for the light generation of the lamp. Besides Xe as the excimer-forming filling component there are other well known excimer-forming filling components like Ne.

Because of the above described drawback due to degradation processes there is the need for alternative phosphors, especially for UV-B lamps, which at least partly overcome the above-mentioned drawbacks and which have a longer lifetime.

### SUMMARY OF THE INVENTION

It is an object of the present invention to provide a discharge lamp which is at least partly able to overcome the above-mentioned drawbacks and especially allows building a discharge lamp with good or improved lighting features together with an increased lifetime for a wide range of applications.

This object is achieved by means of a discharge lamp according to claim 1 of the present invention. Accordingly, a discharge lamp, preferably a low-pressure gas discharge lamp, is provided, said discharge lamp being provided with a discharge vessel comprising a gas filling with a discharge-maintaining composition, wherein at least a part of a wall of the discharge vessel is provided with a luminescent material emitting UV light and comprising Praesodymium(III) as an activator.

The term “activator” in the sense of the present invention especially includes and/or means an impurity present in the given host lattice, in particular Pr(III) ions, which emits radiation upon excitation.

Surprisingly, it has been found that for a wide range of applications within the present invention the use of Praesodymium (Pr) as an activator, which is presented here for the first time, has at least one of the following advantages:

The spectra of the luminescent materials having Pr(III) as an activator have excellent luminescent characteristics and can be used for UV radiation-emitting discharge lamps, especially UV-B radiation-emitting discharge lamps.

Many luminescent materials (and lamps using these materials) have an increased lifetime without deterioration of their emission characteristics.

The materials are readily obtainable and can be used for all types of discharge lamps employed in the field.

The materials used are non-toxic and are therefore usable for a wide range of applications within the present invention.

The materials applied are radiation hard and can thus be used for all types of discharge lamps present in the field.

The materials applied are stable in water, even at a low pH, and organic solvents, and are therefore applicable in many types of suspensions.

Preferably, the discharge lamp is a Xe, Ne, or Xe/Ne excimer discharge lamp and/or preferably a UV-B emitting lamp (i.e. it has at least one peak maximum between 280 and 320 nm).

According to another embodiment of the present invention, the luminescent material comprises a garnet material. The term “garnet material” especially includes and/or means all materials A₃B₅O₁₂ with A and B being suitable trivalent cations (or a mixture of several suitable trivalent cations).

Preferably, the luminescent material is essentially made of a garnet material. The term “essentially” especially includes and/or means ≧95 (wt.) %, preferably ≧98 (wt.) % and most preferably ≧99 (wt.) %.

According to another embodiment of the present invention, the content of Pr(III) in said luminescent material is ≧0 and ≦10 mol % (of the suitable trivalent cations). This has been shown to be advantageous for many applications. Preferably, the content of Pr(III) in said luminescent material is ≧2 and ≦8 mol %, more preferably ≧3,5 and ≦6 mol %.

In case the luminescent material comprises a garnet material, the content of Pr(III) is >0 and ≦10 mol %, preferably ≧2 and ≦8 mol %, more preferably ≧3.5 and ≦6 mol % of the trivalent cation A (i.e. the dodecahedral positions).

Preferably, the luminescent material comprises essentially a material chosen from the group comprising (Y_{1-x-y}Luₓ)₃(Al₁₋ₐGaₐ)₅O₁₂:Pr_{y} or (Lu_{1-x-y}Yₓ)₃(Al₁₋ₐGaₐ)₅O₁₂:Pr_{y} with a, x≧0.0 and ≦1.0 and y>0.0 and ≦0.1. This material has been found to be especially advantageous in many applications for the following reasons:

The materials are easily made and especially stable.

Furthermore, and quite surprisingly, the emission band position of the luminescent material can be easily tuned by the Al/Ga ratio.

According to a preferred embodiment, a is ≧0.0 and ≦0.5. This has shown to be advantageous for many applications because it leads to the emission band being usually in a favorable wavelength area.

According to a preferred embodiment, y is ≧0.02 and ≦0.08, more preferably ≧0.035 and ≦0.06.

According to a preferred embodiment, x is ≦0.8, more preferably ≦0.6.

The present invention furthermore relates to the use of Pr (III) as an activator in UV-B emitting illumination systems.

A system comprising a discharge lamp as described or making use of Pr(III) as described may be used in one or more of the following applications:

equipment for medical therapy

equipment for cosmetic skin treatment (e.g. tanning devices)

water sterilization and/or purification applications, e.g. by the photochemical activation of Cl₂ or ClO₂

chemical reactors, e.g. for the photochemical synthesis of advanced chemical products, e.g. Vitamin D₃

Especially if the present invention is used as or with a luminescent screen it is noted that in these (or other suitable) applications the system might also comprise a second or third UV-B emitting phosphor, e.g. LaPO₄:Ce or SrAl₁₂O₁₉:Ce, to further optimize the lamp spectrum to the action spectrum of the given application.

The aforementioned components, as well as the claimed components and the components to be used in accordance with the invention in the described embodiments, are not subject to any special exceptions with respect to their size, shape, material selection and technical concept, so that the selection criteria known in the pertinent field can be applied without limitations.

### BRIEF DESCRIPTION OF THE DRAWINGS

Additional details, features, characteristics and advantages of the object of the invention are disclosed in the subclaims, the Figures and the following description of the respective Figures and examples, which—in an exemplary fashion—show several embodiments and examples of discharge lamps according to the present invention.

FIG. 1 shows a very schematic cross-sectional view of a discharge lamp according to a first embodiment of the present invention.

FIG. 2 shows the excitation and emission spectrum of a first luminescent material according to the present invention (Example I).

FIG. 3 shows an emission spectrum of a single-component Xe excimer discharge lamp comprising the material of Example I and a standard 290 glass vessel.

FIG. 4 shows a diagram showing the relative output of the lamp of FIG. 3 over time.

FIG. 5 shows the excitation and emission spectrum of a second luminescent material (Example II) according to the present invention.

FIG. 6 shows the excitation and emission spectrum of a third luminescent material (Example III) according to the present invention.

FIG. 7 shows an emission spectrum of a single-component Xe excimer discharge lamp comprising the material of Example III and a quartz glass vessel.

FIG. 8 shows the excitation and emission spectrum of a fourth luminescent material according to the present invention.

FIG. 1 shows a very schematic cross-sectional view of a discharge lamp according to a first embodiment of the present invention. The discharge lamp 10 (which is principally prior art) comprises a glass tube 14 in which a phosphor 12 is provided. This phosphor comprises the luminescent material of the present invention. Furthermore two electrodes (e.g. made of Al) 16 are provided.

### EXAMPLE I

Example I refers to Lu₃Al₅O₁₂:Pr(0.5%), which was made in the following way:

The starting materials Lu₂O₃, Al₂O₃ and Pr₆O₁₁ are dissolved in conc. HNO₃. Then the solvent is removed by evaporation and the remaining powder is fired for 2 h at 600° C. to decompose the nitrates.

Subsequently, the material obtained is powdered and AlF₃ is added as a flux. Afterwards, the powder is annealed for 3 h at 1100° C. in a CO atmosphere, powdered and fired again for 4 h between 1500° and 1700° C. in air. Finally, the obtained powder cake is crushed and the powder is sieved through a 36 μm sieve.

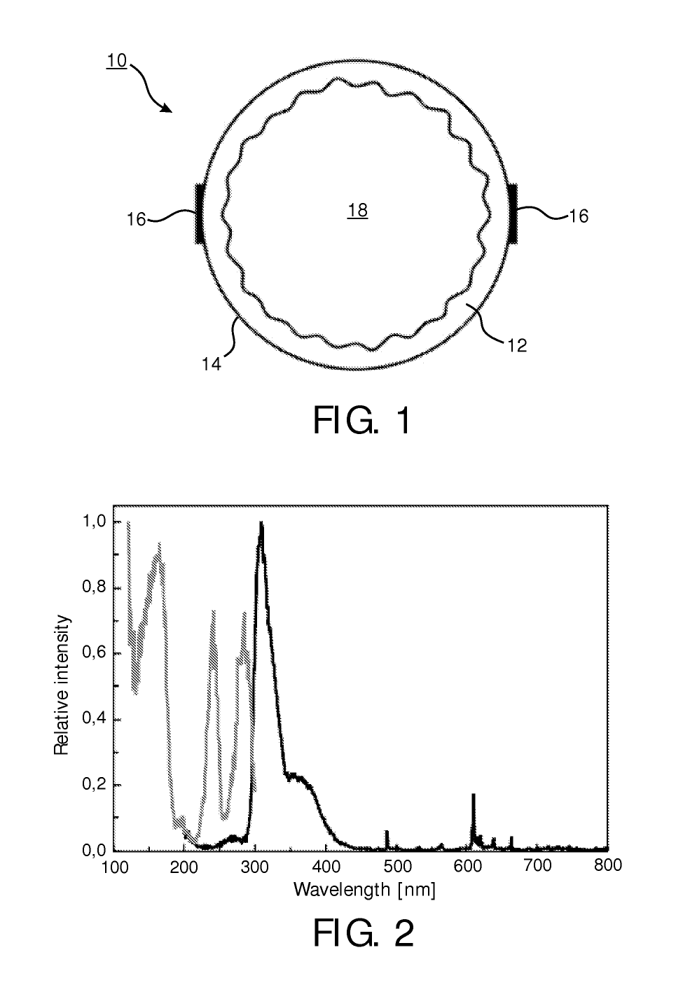

FIG. 2 shows the excitation spectrum (left spectrum) and the emission spectrum (right spectrum) of the material of Example I. It can clearly be seen that this material is an excellent material for use in discharge lamps for UV-B radiation.

Using the material of Example I, a lamp was made in the following way:

Lamp I: Single-component Xe excimer discharge lamp comprising a luminescent layer comprising Lu₃Al₅O₁₂:Pr and a standard 290 glass vessel.

A suspension of MgO nanoparticles is made on a butylacetate basis with nitrocellulose as a binder. The suspension is applied to the inner wall of a standard 290 glass tube by using a flow coat related procedure. Then a suspension of a Lu₃Al₅O₁₂:Pr is prepared on a butylacetate basis with nitrocellulose as a binder. Using a similar flow coat related procedure, the suspension is applied to the inner wall of the precoated lamp tube with a typical phosphor layer weight in the range 2-6 mg/cm². The binder is burned in a standard heating cycle with peak temperatures between 500 and 600° C. The glass tube is filled with Xe, using a thorough pumping cycle. Oxygen impurities have to be strictly excluded, and the glass tube is finally sealed. Typical gas pressures are 200-300 mbar pure Xe. Al-electrodes are attached to the outer side of the tube by means of adhesion or painting. The lamps are typically operated at 5 kV and 25 kHz, using a pulse driving scheme.

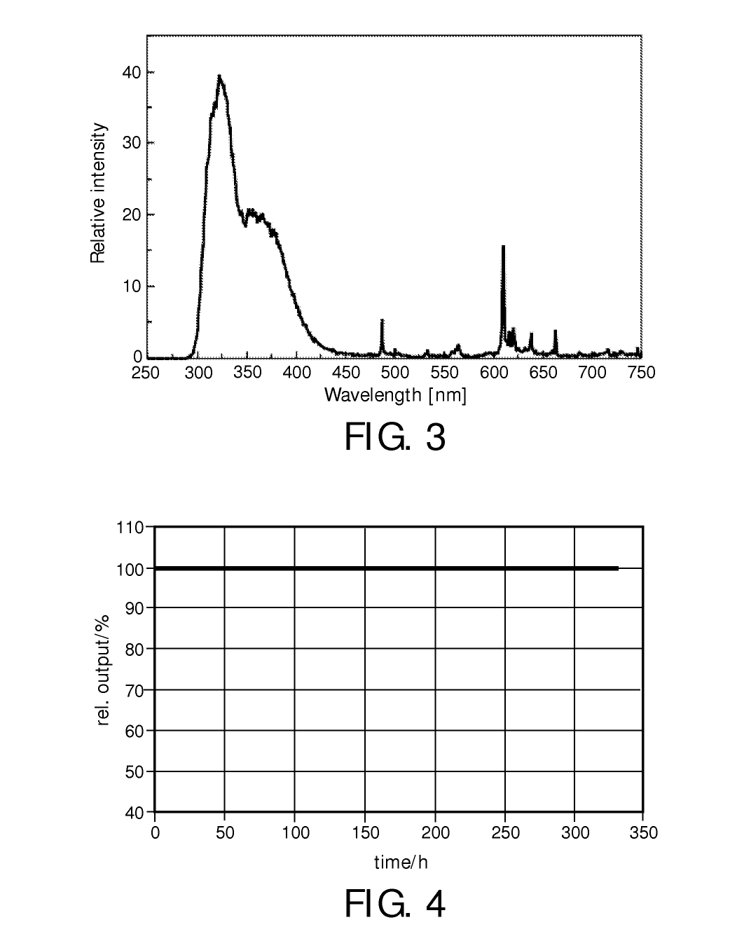

The emission spectrum is determined using an optical spectrum multianalyser and is shown in FIG. 3. It can be seen that the spectrum has a big peak around 325 nm. Therefore, lamps like Lamp I could e.g. be used for tanning devices.

In a further experiment the performance of the lamp over time was investigated. To this end, the relative output of the lamp over time was measured while the lamp was continuously running at a power density of 0.3 W/cm². The diagram is shown in FIG. 4 and shows that even after more than 300 hrs there was no deterioration in the performance of the lamp. This further underlines the possible advantages of using Pr (III) as an activator.

### EXAMPLE II

Example II refers to Lu₃Al₄GaO₁₂:Pr(0.5%), which was made in the following way:

The starting materials Lu₂O₃, Al₂O₃, Ga₂O₃, and Pr₆O₁₁ are dissolved in conc. HNO₃. Then the solvent is removed by evaporation and the remaining powder is fired for 2 h at 600° C. to decompose the nitrates.

Subsequently, the obtained material is powdered and AlF₃ is added as a flux. Afterwards, the powder is annealed for 3 h at 1100° C. in a CO atmosphere, powdered and fired again for 4 h between 1500° and 1700° C. in air. Finally, the obtained powder cake is crushed and the powder is sieved through a 36 μm sieve.

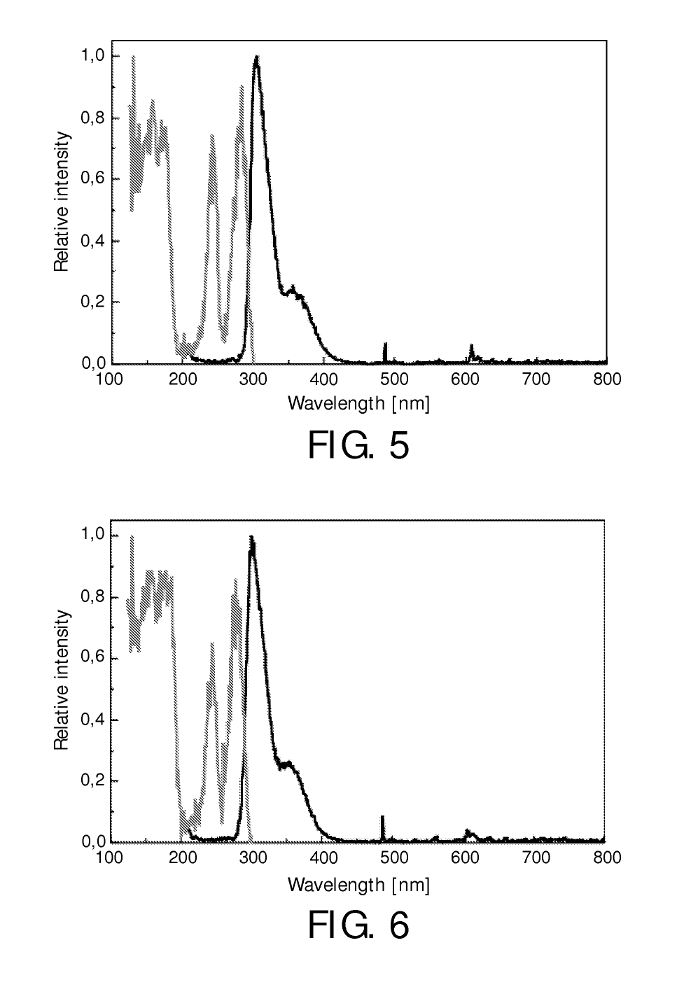

FIG. 5 shows the excitation spectrum (left spectrum) and the emission spectrum (right spectrum) of the material of Example II. It can clearly be seen that this material is an excellent material for use in discharge lamps for UV-B radiation.

### EXAMPLE III

Example III refers to Lu₃Al₃Ga₂O₁₂:Pr(0.5%), which was made in the following way:

The starting materials Lu₂O₃, Al₂O₃, Ga₂O₃, and Pr₆O₁₁ are dissolved in conc. HNO₃. Then the solvent is removed by evaporation and the remaining powder is fired for 2 h at 600° C. to decompose the nitrates.

Subsequently, the obtained material is powdered and AlF₃ is added as a flux. Afterwards, the powder is annealed for 3 h at 1100° C. in a CO atmosphere, powdered and fired again for 4 h between 1500° and 1700° C. in air. Finally, the obtained powder cake is crushed and the powder is sieved through a 36 μm sieve.

FIG. 6 shows the excitation spectrum (left spectrum) and the emission spectrum (right spectrum) of the material of Example III. It can clearly be seen that this material is an excellent material for use in discharge lamps for UV-B radiation.

Using the material of Example III, a lamp was made in the following way:

LAMP II: Single-component Xe excimer discharge lamp comprising a luminescent layer comprising a Lu₃Al₃Ga₂O₁₂:Pr phosphor and a quartz glass vessel.

A suspension of MgO nanoparticles is made on a butylacetate basis with nitrocellulose as a binder. The suspension is applied to the inner wall of a quartz tube by using a flow coat related procedure. Then a suspension of Lu₃Al₃Ga₂O₁₂:Pr is prepared on a butylacetate basis with nitrocellulose as a binder. Using a similar flow coat related procedure, the suspension is applied to the inner wall of the precoated lamp tube with a typical phosphor layer weight in the range 1-10 mg/cm². The binder is burned in a standard heating cycle with peak temperatures between 500 and 600° C. The glass tube is filled with Xe using a thorough pumping cycle. Oxygen impurities have to be strictly excluded, and finally the glass tube is sealed. Typical gas pressures are 200-300 mbar pure Xe. Al-electrodes are attached to the outer side of the tube by means of adhesion or painting. The lamps are typically operated at 5 kV and 25 kHz using a pulse driving scheme. The emission spectrum is determined using an optical spectrum multianalyser.

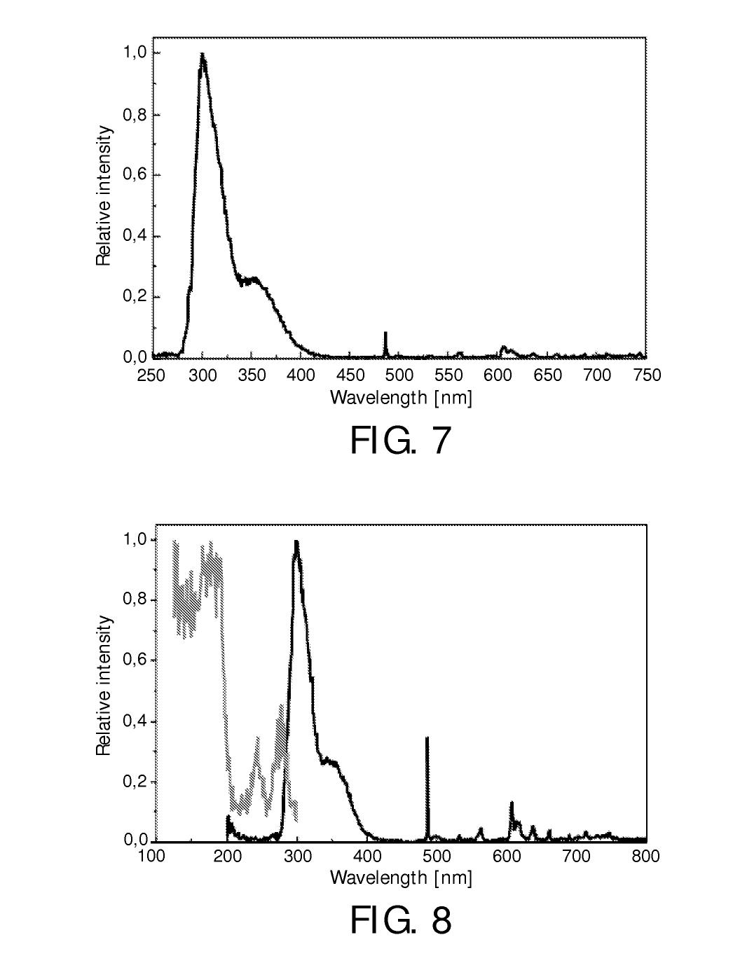

The emission spectrum is determined using an optical spectrum multianalyser and is shown in FIG. 7. It can be seen that the spectrum has a big peak around 315 nm. This is e.g. suitable for Vitamin D production in skin or photochemical reactors and for the photochemical cleavage of Cl₂ or ClO₂, which makes this lamp very useful especially for these applications.

### EXAMPLE IV

Example IV refers to Lu₃Al_{2.5}Ga_{2.5}O₁₂:Pr(0.5%) which was made in the following way:

The starting materials Lu₂O₃, Al₂O₃, Ga₂O₃, and Pr₆O₁₁ are dissolved in conc. HNO₃. Then the solvent is removed by evaporation and the remaining powder is fired for 2 h at 600° C. to decompose the nitrates.

Subsequently, the obtained material is powdered and AlF₃ is added as a flux. Afterwards, the powder is annealed for 3 h at 1100° C. in a CO atmosphere, powdered and fired again for 4 h between 1500° and 1700° C. in air. Finally, the obtained powder cake is crushed and the powder is sieved through a 36 μm sieve.

FIG. 8 shows the excitation spectrum (left spectrum) and the emission spectrum (right spectrum) of the material of Example IV. It can clearly be seen that this material is an excellent material for use in discharge lamps for UV-B radiation.

The particular combinations of elements and features in the above detailed embodiments are exemplary only; the interchanging and substitution of these teachings with other teachings in this patent application and the patents/applications incorporated herein by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The invention's scope is defined in the following claims and equivalents thereto. Furthermore, reference signs used in the description and claims do not limit the scope of the invention as claimed.

1. Discharge lamp, preferably a low-pressure gas discharge lamp, provided with a discharge vessel comprising a gas filling having a discharge-maintaining composition, wherein at least a part of a wall of the discharge vessel is provided with a luminescent material emitting UV light and comprising Praesodymium (III) as an activator.

2. Discharge lamp according to claim 1, wherein the discharge lamp is a Xe, Ne, or Xe/Ne excimer discharge lamp.

3. Discharge lamp according to claim 1, wherein the luminescent material comprises a garnet material.

4. Discharge lamp according to claim 1, wherein the content of Pr(III) in said luminescent material is >0 and ≦10 mol %.

5. Discharge lamp according to claim 1, wherein the luminescent Material comprises essentially a material from the group comprising (Y_{1-x-y}Luₓ)₃(Al₁₋ₐGaₐ)₅O₁₂:Pr_{y} or (Lu_{1-x-y}Yₓ)₃(Al₁₋ₐGaₐ)₅O₁₂:Pr_{y}, with a, x≧0.0 and ≦1.0 and y>0.0 and ≦0.1.

6. Discharge lamp according to claim 1, wherein the luminescent material comprises essentially a material from the group comprising (Y_{1-x-y}Luₓ)₃(Al₁₋ₐGaₐ)₅O₁₂:Pr_{y} or (Lu_{1-x-y}Yₓ)₃(Al₁₋ₐGaₐ)₅O₁₂:Pr_{y}, with a≧0.0 and ≦0.5, x≧0.0 and ≦1.0 and y>0.0 and ≦0.1.

7. Use of Pr(III) as an activator in UV-B emitting illumination systems.

8. A system comprising a discharge lamp according to claim 1, the system being used in one or more of the following applications:
equipment for medical therapy
equipment for cosmetic skin treatment (e.g. tanning devices)
water sterilization and/or purification applications, e.g. by the photochemical activation of Cl₂ or ClO₂
chemical reactors, e.g. for the photochemical synthesis of advanced chemical products, e.g. Vitamin D₃
